(19) United States
(12) Patent Application Publication
(10) Pub. No.: US 2012/0083782 A1
(43) Pub. Date: Apr. 5, 2012

(54) ELECTROSURGICAL APPARATUS WITH LOW WORK FUNCTION ELECTRODE

(75) Inventors: Kenneth R. Stalder, Redwood City, CA (US); Jean Woloszko, Austin, TX (US); Richard Christensen, San Francisco, CA (US)

(73) Assignee: ArthroCare Corporation, Austin, TX (US)

(21) Appl. No.: 12/897,311

(22) Filed: Oct. 4, 2010

(51) Int. Cl. A61B 18/14

(52) U.S. Cl. 606/41

(57) ABSTRACT

An electrosurgical apparatus includes an active electrode with a low-work function coating to improve ablation performance. Low-work function coatings include compounds of alkali metals and alkali earth metals. Additionally, the active electrode may include various micro-structures or asperities or nano-structures or asperities. An array of carbon nanotubes may be aligned and secured on the active electrode. A return electrode comprises a high-work function coating to suppress electrical discharge activity on the return electrode.

### FIELD OF THE INVENTION

The present invention relates to electrosurgical apparatuses for ablating tissue. More particularly, the present invention relates to electrosurgical apparatuses having electrodes with enhanced work function and electrical discharge characteristics.

### BACKGROUND OF THE INVENTION

The field of electrosurgery includes a number of loosely related surgical techniques which have in common the application of electrical energy to modify the structure or integrity of patient tissue. Electrosurgical procedures usually operate through the application of very high frequency currents to cut or ablate tissue structures, where the operation can be monopolar or bipolar. Monopolar techniques rely on a separate electrode for the return of RF current that is placed away from the surgical site on the body of the patient, and where the surgical device defines only a single electrode pole that provides the surgical effect. Bipolar devices include both an active electrode and a return electrode on the device so that some portion of the current flowing in the system does not flow through patient tissue.

Electrosurgical procedures and techniques are particularly advantageous since they generally reduce patient bleeding and trauma associated with cutting operations. Additionally, electrosurgical ablation procedures, where tissue surfaces and volume may be reshaped, cannot be duplicated through other treatment modalities. Radiofrequency (RF) energy is extensively used during arthroscopic procedures because it provides efficient tissue resection and coagulation and relatively easy access to the target tissues through a portal or cannula.

RF electrosurgical devices, however, are not entirely problem-free. One area of concern involves the consistency of the electrical discharges from the electrodes (namely, electrode “firing”). On occasion, electrode firing is inconsistent. The problems associated with electrode firing can occur in connection with the active electrode or the return electrode. In the case of the active electrode, it may not consistently fire. In the case of the return electrode, it may fire even though it is not supposed to do so. Return electrode firing suppresses plasma activity on active electrodes, and results in inconsistent ablation and coagulation. Regardless of whether the inconsistent plasma activity and electrical discharge arises from the active electrode or the return electrode, inconsistent plasma activity is undesirable because it decreases the performance of the ablation procedure. Ablation procedures may take longer than anticipated. Electrodes may not fire properly when needed to do so during a procedure.

Therefore, an electrosurgical apparatus having improved electrical discharge characteristics is still desired.

### SUMMARY OF THE INVENTION

An electrosurgical apparatus for treating tissue at a target site includes a shaft having a distal end and a proximal end. An active electrode is disposed near the distal end. The active electrode features a low work function surface. When a voltage difference is applied between the active electrode and a return electrode in the presence of an electrically conductive fluid a current path is formed there between. The low-work function surface enhances electrical discharge activity of the electrosurgical apparatus.

In another embodiment of the invention, micro-structures or asperities and nano-structures or asperities are disposed on the surface of the active electrode. The nano-structures or asperities in one embodiment have a tubular shape.

In another embodiment of the invention, the nanotubes are carbon nanotubes and are disposed in an aligned formation or array.

In another embodiment of the invention, the active electrode includes a low work function surface and is comprised of compound that is biocompatible. In another embodiment of the invention, the compound is insoluble in saline. In another embodiment of the invention, the surface of the active electrode is comprised of a compound such that the low-work function has a work function of less than or equal to about 2-3 eV. In another embodiment of the invention, the low-work function surface comprises a layer of an alkali metal or alkaline earth metal. In another embodiment of the invention, the surface layer of the active electrode includes barium oxide.

In another embodiment of the invention, the active electrode is doped or infused with a material such as a rare earth element, thereby forming a low-work function surface.

In another embodiment of the invention, the electrosurgical apparatus comprises a return electrode and the return electrode includes a high-work function surface for decreasing discharge activity. The return electrode in one embodiment of the invention is disposed along the shaft of the apparatus and proximal to the active electrode. In another embodiment of the invention, the high-work function material comprises a compound selected from the group consisting of platinum (work function ≈5.65 eV), iridium, (work function ≈5.7 eV), osmium (work function ≈5.9 eV), palladium (work function ≈5.2 eV) or gold (work function ≈5.1 eV). The high work function return electrode described herein may be incorporated into an electrosurgical apparatus with or without optimizing the design of the active electrode.

In another embodiment of the present invention, an electrosurgical apparatus for treating tissue at a target site comprises a shaft having a distal end and a proximal end and an active electrode disposed near the distal end. The active electrode comprises a surface comprising at least one of micro-structures or asperities and nano-structures or asperities to enhance electrical discharge activity. When a voltage difference is applied between the active electrode and the return electrode in the presence of an electrically conductive fluid a current path is formed there between. The active electrode may additionally be enhanced by depositing a low work function layer or coating on the active electrode. The nano or micro structures or asperities may include tubular shapes and in one embodiment, the nano-structures include an aligned array of carbon nanotubes. The return electrode may comprise a high work function surface layer. The return electrode may be disposed on the shaft proximal to the active electrode.

The description, objects and advantages of the present invention will become apparent from the detailed description to follow, together with the accompanying drawings.

### BRIEF DESCRIPTION OF THE DRAWINGS

FIG. 1 is a perspective view of an electrosurgical system including an electrosurgical probe and electrosurgical power supply;

FIG. 2 is side view of an electrosurgical probe;

FIG. 3 is a cross-sectional view of the electrosurgical probe of FIG. 2;

FIG. 4A is a perspective view of an embodiment of the active electrode for the probe of FIGS. 1 and 2;

FIG. 4B is a detailed view of the distal tip of the electrosurgical probe of FIGS. 1 and 2 incorporating the active screen electrode of FIG. 4A;

FIG. 5 is a graph of the Fowler-Nordheim Field Emission versus Work-Function;

FIG. 6 is a partial section of an active electrode comprising an array of nanotubes; and

FIG. 7 is an illustration of a distal end of an electrosurgical probe ablating tissue.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The electrosurgical apparatus or device of the present invention may have a variety of configurations. However, one variation of the device employs a treatment device using Coblation® technology (ArthroCare Corporation, Austin, Tex.).

The assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the tip of the active electrode(s) and the target tissue. The electrically conductive fluid may be a liquid or gas, such as isotonic saline, blood, extracelluar or intracellular fluid, delivered to, or already present at, the target site, or a viscous fluid, such as a gel, applied to the target site.

When the conductive fluid is heated enough such that atoms vaporize off the surface faster than they recondense, a gas layer, or vapor layer is formed. When the gas or vapor is formed near the electrode the electric field in the gas or vapor layer is very much enhanced because the electrical conductivity of the gas or vapor is very much lower than the electrical conductivity of the conductive fluid. Electrons emitted from the electrode are accelerated in this enhanced electric field and achieve sufficient energy to ionize molecules in the gas or vapor phase, thus producing more free electrons which also may be accelerated, thus causing the ionization level in the gas or vapor layer to increase substantially. This process forms an ionized gas, or plasma (the so-called “fourth state of matter”). Generally speaking, plasmas may be formed by a variety of means, including ionizing the gas by driving an electric current through it, or by shining electromagnetic waves through the gas, or by a number of other means. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the molecules in the gas or vapor layer becomes sufficiently low (i.e., less than approximately 10²⁰ atoms/cm³), the electron mean free path increases to enable subsequently injected electrons to cause impact ionization within the vapor layer. Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are significantly more energetic than the ions. Thus, the electrons, which are carried away from the tissue towards the return electrode, carry most of the plasma's energy with them, allowing them to break apart the tissue molecules in a substantially non-thermal manner. Moreover, many chemical reactions can be produced by these high energy electrons, which can produce chemically-active neutral species such at hydroxyl radicals (OH) or atomic hydrogen (H), both of which are known to react vigorously with organic molecules. The production of chemically active species, along with the reactions of very energetic electrons and ions with other less energetic molecules is known as a nonequilibrium, or non-thermal process, since the interacting particles have substantially different mean energies (temperatures).

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of this phenomena can be found in commonly assigned U.S. Pat. No. 5,697,882, the complete disclosure of which is incorporated herein by reference.

In some applications of the Coblation® technology, high frequency (RF) electrical energy is applied in an electrically conducting media environment to shrink or remove (i.e., resect, cut, or ablate) a tissue structure and to seal transected vessels within the region of the target tissue. Coblation® technology is also useful for sealing larger arterial vessels, e.g., on the order of about 1 mm in diameter. In such applications, a high frequency power supply is provided having an ablation mode, wherein a first voltage is applied to an active electrode sufficient to effect molecular dissociation or disintegration of the tissue, and a coagulation mode, wherein a second, lower voltage is applied to an active electrode (either the same or a different electrode) sufficient to heat, shrink, and/or achieve hemostasis of severed vessels within the tissue.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the free electrons. Since different tissue structures have different molecular bonds, the Coblation® device may be configured to produce energy sufficient to break the molecular bonds of certain tissue but insufficient to break the molecular bonds of other tissue. For example, fatty tissue (e.g., adipose) has double bonds that require an energy level substantially higher than 4 eV to 5 eV (typically on the order of about 8 eV) to break. Accordingly, the Coblation® technology generally does not ablate or remove such fatty tissue; however, it may be used to effectively ablate cells to release the inner fat content in a liquid form. Of course, factors may be changed such that these double bonds can also be broken in a similar fashion as the single bonds (e.g., increasing voltage or changing the electrode configuration to increase the current density at the electrode tips). A more complete description of this phenomena can be found in commonly assigned U.S. Pat. Nos. 6,355,032, 6,149,120 and 6,296,136, the complete disclosures of which are incorporated herein by reference.

The active electrode(s) of a Coblation® device may be supported within or by an inorganic insulating support positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

In one example of a Coblation® device for use with the embodiments disclosed herein, the return electrode of the device is typically spaced proximally from the active electrode(s) a suitable distance to avoid electrical shorting between the active and return electrodes in the presence of electrically conductive fluid. In many cases, the distal edge of the exposed surface of the return electrode is spaced about 0.5 mm to 25 mm from the proximal edge of the exposed surface of the active electrode(s), preferably about 1.0 mm to 5.0 mm. Of course, this distance may vary with different voltage ranges, conductive fluids, and depending on the proximity of tissue structures to active and return electrodes. The return electrode will typically have an exposed length in the range of about 1 mm to 20 mm.

A Coblation® treatment device for use according to the present embodiments may use a single active electrode or an array of active electrodes spaced around the distal surface of a catheter or probe. In the latter embodiment, the electrode array usually includes a plurality of independently current-limited and/or power-controlled active electrodes to apply electrical energy selectively to the target tissue while limiting the unwanted application of electrical energy to the surrounding tissue and environment resulting from power dissipation into surrounding electrically conductive fluids, such as blood, normal saline, and the like. The active electrodes may be independently current-limited by isolating the terminals from each other and connecting each terminal to a separate power source that is isolated from the other active electrodes. Alternatively, the active electrodes may be connected to each other at either the proximal or distal ends of the catheter to form a single wire that couples to a power source.

In one configuration, each individual active electrode in the electrode array is electrically insulated from all other active electrodes in the array within the instrument and is connected to a power source which is isolated from each of the other active electrodes in the array or to circuitry which limits or interrupts current flow to the active electrode when low resistivity material (e.g., blood, electrically conductive saline irrigant or electrically conductive gel) causes a lower impedance path between the return electrode and the individual active electrode. The isolated power sources for each individual active electrode may be separate power supply circuits having internal impedance characteristics which limit power to the associated active electrode when a low impedance return path is encountered. By way of example, the isolated power source may be a user selectable constant current source. In this embodiment, lower impedance paths will automatically result in lower resistive heating levels since the heating is proportional to the square of the operating current times the impedance. Alternatively, a single power source may be connected to each of the active electrodes through independently actuatable switches, or by independent current limiting elements, such as inductors, capacitors, resistors and/or combinations thereof. The current limiting elements may be provided in the instrument, connectors, cable, controller, or along the conductive path from the controller to the distal tip of the instrument. Alternatively, the resistance and/or capacitance may occur on the surface of the active electrode(s) due to oxide layers which form on selected active electrodes (e.g., titanium or a resistive coating on its surface, such as titanium oxide).

The Coblation® device is not limited to electrically isolated active electrodes, or even to a plurality of active electrodes. For example, the array of active electrodes may be connected to a single lead that extends through the catheter shaft to a power source of high frequency current.

The voltage difference applied between the return electrode(s) and the active electrode(s) will be at high or radio frequency, typically between about 5 kHz and 20 MHz, usually being between about 30 kHz and 2.5 MHz, preferably being between about 50 kHz and 500 kHz, often less than 350 kHz, and often between about 100 kHz and 200 kHz. In some applications, applicant has found that a frequency of about 100 kHz is useful because the tissue impedance is much greater at this frequency. In other applications, such as procedures in or around the heart or head and neck, higher frequencies may be desirable (e.g., 400-600 kHz) to minimize low frequency current flow into the heart or the nerves of the head and neck.

The RMS (root mean square) voltage applied will usually be in the range from about 5 volts to 1000 volts, preferably being in the range from about 10 volts to 500 volts, often between about 150 volts to 400 volts depending on the active electrode size, the operating frequency and the operation mode of the particular procedure or desired effect on the tissue (i.e., contraction, coagulation, cutting or ablation.)

Typically, the peak-to-peak voltage for ablation or cutting with a square wave form will be in the range of 10 volts to 2000 volts and preferably in the range of 100 volts to 1800 volts and more preferably in the range of about 300 volts to 1500 volts, often in the range of about 300 volts to 800 volts peak to peak (again, depending on the electrode size, number of electrons, the operating frequency and the operation mode). Lower peak-to-peak voltages will be used for tissue coagulation, thermal heating of tissue, or collagen contraction and will typically be in the range from 50 to 1500, preferably 100 to 1000 and more preferably 120 to 400 volts peak-to-peak (again, these values are computed using a square wave form). Higher peak-to-peak voltages, e.g., greater than about 800 volts peak-to-peak, may be desirable for ablation of harder material, such as bone, depending on other factors, such as the electrode geometries and the composition of the conductive fluid.

As discussed above, the voltage is usually delivered in a series of voltage pulses or alternating current of time varying voltage amplitude with a sufficiently high frequency (e.g., on the order of 5 kHz to 20 MHz) such that the voltage is effectively applied continuously (as compared with, e.g., lasers claiming small depths of necrosis, which are generally pulsed about 10 Hz to 20 Hz). In addition, the duty cycle (i.e., cumulative time in any one-second interval that energy is applied) is on the order of about 50% for the present invention, as compared with pulsed lasers which typically have a duty cycle of about 0.0001%.

The preferred power source may deliver a high frequency current selectable to generate average power levels ranging from several milliwatts to tens of watts per electrode, depending on the volume of target tissue being treated, and/or the maximum allowed temperature selected for the instrument tip. The power source allows the user to select the voltage level according to the specific requirements of a particular neurosurgery procedure, cardiac surgery, arthroscopic surgery, dermatological procedure, ophthalmic procedures, open surgery or other endoscopic surgery procedure. For cardiac procedures and potentially for neurosurgery, the power source may have an additional filter, for filtering leakage voltages at frequencies below 100 kHz, particularly frequencies around 60 kHz. Alternatively, a power source having a higher operating frequency, e.g., 300 kHz to 600 kHz may be used in certain procedures in which stray low frequency currents may be problematic. A description of one suitable power source can be found in commonly assigned U.S. Pat. Nos. 6,142,992 and 6,235,020, the complete disclosure of both patents are incorporated herein by reference for all purposes.

The power source may be current limited or otherwise controlled so that undesired heating of the target tissue or surrounding (non-target) tissue does not occur. Current limiting inductors can be placed in series with each independent active electrode, where the inductance of the inductor is in the range of 10 μH to 50,000 μH, depending on the electrical properties of the target tissue, the desired tissue heating rate and the operating frequency. Alternatively, capacitor-inductor (LC) circuit structures may be employed, as described previously in U.S. Pat. No. 5,697,909, the complete disclosure of which is incorporated herein by reference. Additionally, current-limiting resistors may be selected. Preferably, these resistors will have a large positive temperature coefficient of resistance so that, as the current level begins to rise for any individual active electrode in contact with a low resistance medium (e.g., saline irrigant or blood), the resistance of the current limiting resistor increases significantly, thereby minimizing the power delivery from said active electrode into the low resistance medium (e.g., saline irrigant or blood). Moreover, other treatment modalities (e.g., laser, chemical, other RF devices, etc.) may be used in the inventive method either in place of the Coblation® technology or in addition thereto.

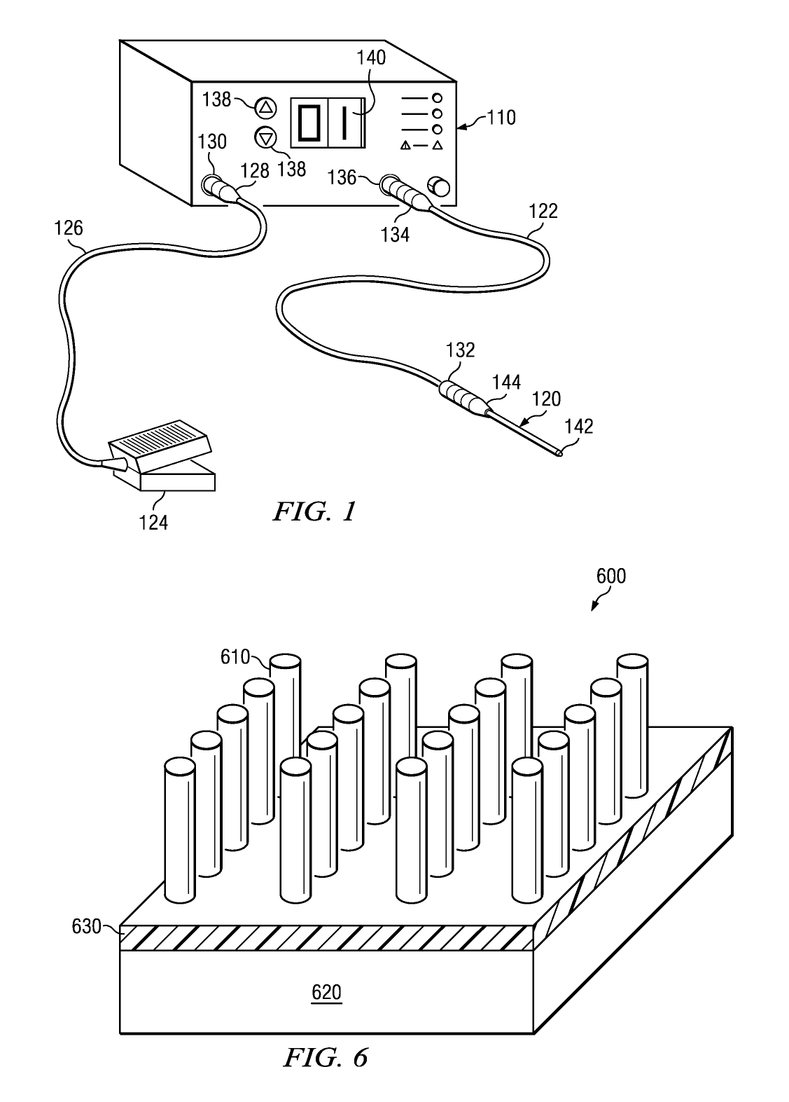

Referring now to FIG. 1, an exemplary electrosurgical system for resection, ablation, coagulation and/or contraction of tissue will now be described in detail. As shown, certain embodiments of the electrosurgical system generally include an electrosurgical probe 120 connected to a power supply 110 for providing high frequency voltage to one or more electrode terminals on probe 120. Probe 120 includes a connector housing 144 at its proximal end, which can be removably connected to a probe receptacle 132 of a probe cable 122. In an alternative embodiment the probe 120 and cable 122 are integrated into one assembly.

With respect to FIG. 1, the proximal portion of cable 122 has a connector 134 to couple probe 120 to power supply 110 at receptacle 136. Power supply 110 has an operator controllable voltage level adjustment 138 to change the applied voltage level, which is observable at a voltage level display 140. Power supply 110 also includes one or more foot pedals 124 and a cable 126 which is removably coupled to a receptacle 130 with a cable connector 128. The foot pedal 124 may also include a second pedal (not shown) for remotely adjusting the energy level applied to electrode terminals 142, and a third pedal (also not shown) for switching between an ablation mode and a coagulation mode.

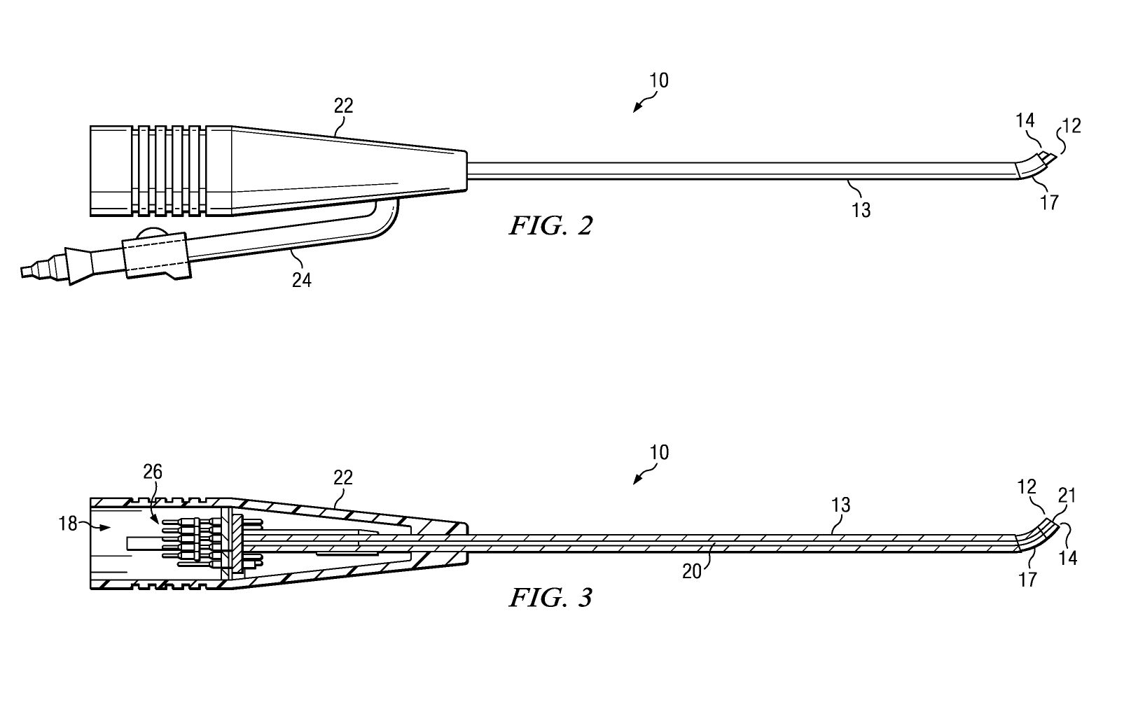

Referring now to FIG. 2, an electrosurgical probe 10 includes an elongate shaft 13 which may be flexible or rigid, a handle 22 coupled to the proximal end of shaft 13 and an electrode support member 14 coupled to the distal end of shaft 13. Probe 10 includes an active electrode terminal 12 disposed on the distal tip of shaft 13. Active electrode 12 may be connected to an active or passive control network within a power supply and controller 110 (see FIG. 1) by means of one or more insulated electrical connectors (not shown). The active electrode 12 is electrically isolated from a common or return electrode 17 which is disposed on the shaft proximally of the active electrode 12, preferably being within 1 mm to 25 mm of the distal tip. Proximally from the distal tip, the return electrode 17 is shown generally concentric with the shaft of the probe 10. The support member 14 is positioned distal to the return electrode 17 and may be composed of an electrically insulating material such as epoxy, plastic, ceramic, glass or the like. Support member 14 may extend from the distal end of shaft 13 (usually about 1 to 20 mm) and provides support for active electrode 12.

Referring now to FIG. 3, probe 10 may further include a suction lumen 20 for aspirating excess fluids, bubbles, tissue fragments, and/or products of ablation from the target site. Suction lumen 20 extends through support member 14 to a distal opening 21, and extends through shaft 13 and handle 22 to an external connector 24 (see FIG. 2) for coupling to a vacuum source. Typically, the vacuum source is a standard hospital pump that provides suction pressure to connector 24 and suction lumen 20. Handle 22 defines an inner cavity 18 that houses electrical connections 26 and provides a suitable interface for electrical connection to power supply/controller 110 via an electrical connecting cable 122 (see FIG. 1).

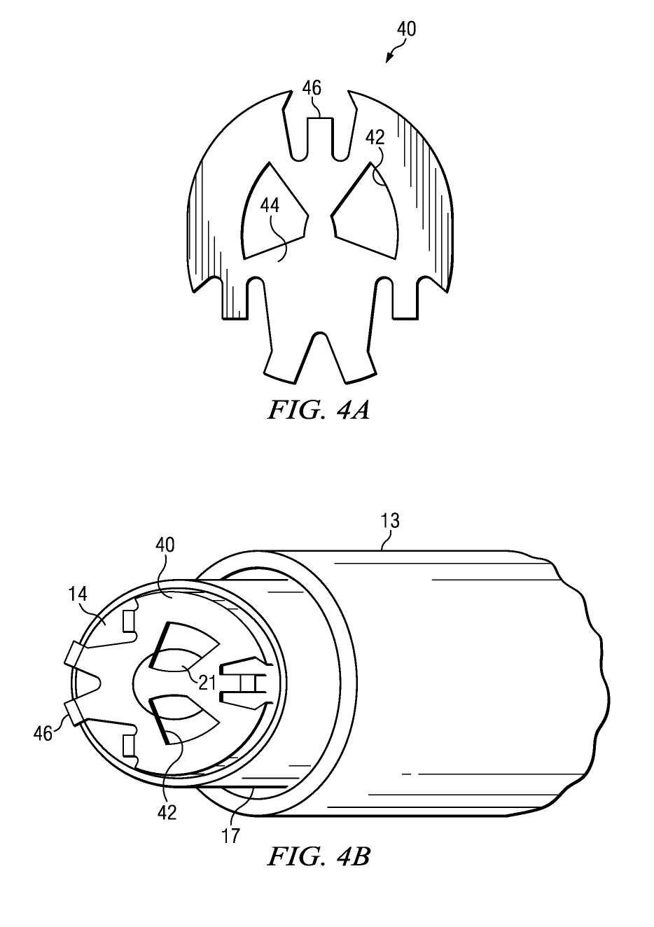

In certain embodiments, active electrode 12 may comprise an active screen electrode 40. Screen electrode 40 may have a variety of different shapes, such as the shapes shown in FIGS. 4A and 4B. Electrical connectors (e.g., wire conductors) extend from connections 26 through shaft 13 to screen electrode 40 to electrically couple the active screen electrode 40 to the high frequency power supply 110 (see FIG. 1).

Screen electrode 40 may comprise a conductive material, such as tungsten, titanium, molybdenum, platinum, or the like. Screen electrode 40 may have a diameter in the range of about 0.5 to 8 mm, preferably about 1 to 4 mm, and a thickness of about 0.05 to about 2.5 mm, preferably about 0.1 to 1 mm. Screen electrode 40 may comprise a plurality of apertures 42 configured to rest over the distal opening 21 of suction lumen 20. Apertures 42 are designed to allow for the passage of aspirated excess fluids, bubbles, and gases from the ablation site and are typically large enough to allow ablated tissue fragments to pass through into suction lumen 20. As shown, screen electrode 40 has a generally irregular shape which increases the edge to surface-area ratio of the screen electrode 40. A large edge to surface-area ratio increases the ability of screen electrode 40 to initiate and maintain a plasma layer in conductive fluid because the edges generate higher current densities, which a large surface area electrode tends to dissipate power into the conductive media. Additional electrode enhancements are discussed further herein.

In the embodiment shown in FIGS. 4A and 4B, screen electrode 40 includes a body 44 that rests over insulative support member 14 and the distal opening 21 to suction lumen 20. Screen electrode 40 is shown having at least five tabs 46 that may rest on, be secured to, and/or be embedded in insulative support member 14. In certain embodiments, electrical connectors extend through insulative support member 14 and are coupled (i.e., via adhesive, braze, weld, or the like) to one or more of tabs 46 in order to secure screen electrode 40 to the insulative support member 14 and to electrically couple screen electrode 40 to power supply 110 (see FIG. 1). Preferably, screen electrode 40 forms a substantially planar tissue treatment surface for smooth resection, ablation, and sculpting of the meniscus, cartilage, and other soft tissues. In reshaping cartilage and meniscus, the physician often desires to smooth the irregular, ragged surface of the tissue, leaving behind a substantially smooth surface. For these applications, a macroscopically or substantially planar screen electrode treatment surface is preferred.

In one embodiment of the invention, electrodes are enhanced by modifying the work function of the electrode surface (by work function, it is meant how much energy is required to liberate an electron from the electrode). Example features and aspects to enhance electrical discharge characteristics and subsequent plasma activity of the electrodes include one or a combination of the following: 1) low-work function materials coated on the active electrodes; 2) micro or nano-structured surfaces to improve electric field emission of the active electrodes, and 3) high work function coatings on return electrodes to suppress electrical discharge activity and plasma production on those surfaces.

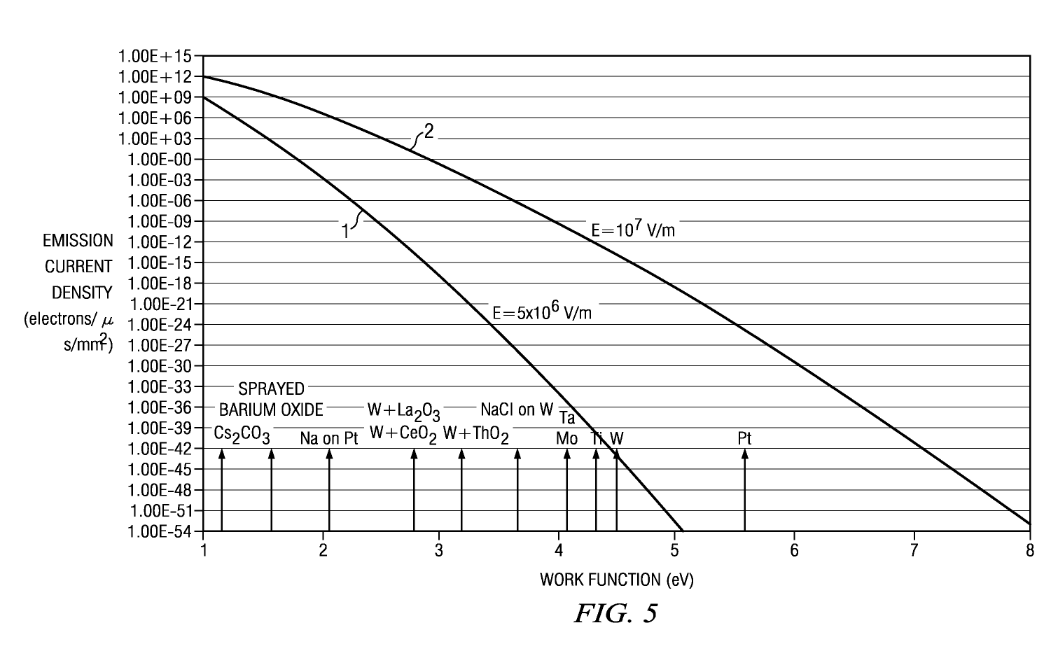

Without intending to be bound by theory, it is thought that the emission of electrons from both active and return electrodes is responsible for current to flow in the circuit of an electrosurgical apparatus. Since the electrodes are always rather relatively cool, the electron emission from either electrode into a surrounding vapor layer, which has an enhanced electric field therein, is believed to be due to a cold emission process, as opposed to a thermionic process. Electron emission in such situations is frequently described as a field emission process, since the electrons are pulled out of the metal electrodes by the external electric field. The electric field strength in a field emission process is very large (>1 MV/m). Field strengths of this magnitude or greater facilitate electron emission by tunneling processes, generally referred to in the literature as Fowler-Nordheim emission and may be tremendously affected by work function as shown in FIG. 5 discussed below.

FIG. 5 is a Fowler-Nordheim graph of emission current density versus work function for various electric field curves, namely, curve 1 (5×10⁶ V^{/}m) or curve 2 (10⁷ V^{/m}). A number of compounds are shown along the work function axis. As can be observed from FIG. 5, modifying the work function of the electrode from, e.g., clean Pt to Sprayed Barium Oxide results in a tremendous increase in emission current density and consequently, performance of the corresponding electrosurgical device.

Additionally, in the case of tungsten (chemical symbol W of FIG. 5), many orders of magnitude improvement of the electron emission can be achieved by coating or otherwise providing the electrode with other compounds such as cerium oxide (CeO₂) or other compounds such as barium oxide to lower the electrode work function. The present invention includes application of various compounds to the surface of the metal electrode, or otherwise incorporates compounds into the metal electrode (e.g., by doping or another alloying process) to significantly alter its work function.

In connection with the active electrode, in one embodiment, the work function is lowered by applying thin layers of various compounds on the electrode surface. A preferred range of values for the work-function of the active electrode surface is 1 to 4 eV and more preferably 1 to 3 eV. This is a meaningful difference from an ordinary active electrode which may have a work function in the range of 4 to 6 eV or more.

Examples of compounds that may be deposited on the electrode to lower the work function of the active electrode include oxides, carbonates, or other forms of alkali metals (group 1 of the periodic table) or alkaline earth metals (group 2 of the periodic table). Preferably, the compound is robust and durable, biocompatible, and insoluble in a saline environment. An example of a preferred compound is barium oxide.

The compounds may be added to the active electrode using various techniques including but not limited to electrochemical deposition, direct growth of films and nanotubes using electrochemical, electropolymerization, dielectrophoresis, sputtering, anodic oxidation, deposition of crown ethers, dip-coating, spin-coating, polymer wrapping, laser-ablation coating, electrolytic deposition, sintering, alkali metal or alkaline earth intercalation, or chemical vapor deposition methods.

Additionally, in an alternative embodiment, the electrode may be doped or infused with another material to modify its work function. Doping the metals used in active electrodes with certain elements, for example, lanthanum, cerium, neodymium, and other rare-earth elements, can significantly reduce the work function of the metallic electrode. A non-limiting example of a compound for use in the present invention is tungsten doped with 2% cerium oxide.

Unlike the active electrode, the work function of the return electrode is desirably increased so as to prohibit electrical discharge or firing. Preferably the work function of the return electrode is increased to 5 eV or more. Examples of coatings for the return electrode include platinum or platinum-iridium. The coatings may be deposited on the electrode in accordance with the manufacturing processes mentioned above. A preferred compound for the untreated return electrode is stainless steel.

Another embodiment of the invention includes modification of the electric field to increase emission. In one embodiment of the invention, the surface of the active electrode includes texture, surface roughness, structures, asperities, and or pits. The field-enhancement structures or asperities on the electrodes serve to locally increase the electric field on the surface of the electrode. Field enhancement structures or asperities may take a wide variety of shapes including but not limited to cones, tubes, balls, or pillars. The size of the structures preferably ranges from tens of nanometers to hundreds of micrometers.

In one embodiment the structures or asperities are aligned preferentially to enhance the local electric field at the metal surface. In another embodiment low work function materials are combined with field enhancement structures or asperities to significantly improve the electron emission characteristics of the electrode.

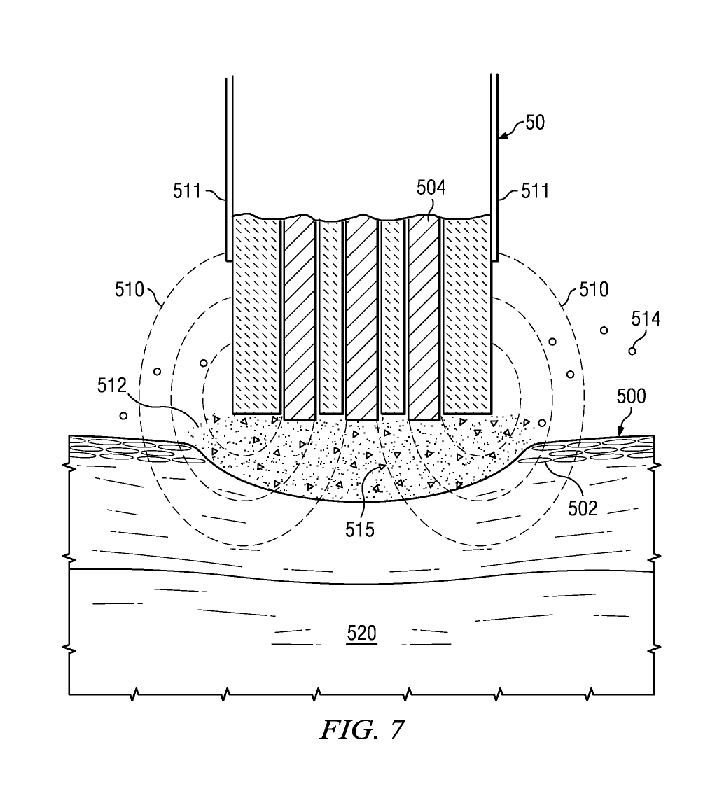

With reference to FIG. 6, an active electrode 600 includes an array of nanotubes 610. Nanotubes are deposited or attached to the electrode substrate. Although the nanotubes 610 are shown here in a regular array, it is to be understood that the nanotubes could be randomly dispersed over the surface of the electrode, or could have regions of enhanced density. The structures need not all be aligned perpendicularly as shown, nor do they have to be of uniform diameter or length.

As mentioned herein the nanotubes and similar structures can increase emissions. An exemplary nanotube is a carbon nanotube. However, the nanostructures or asperities need not be pure carbon nanotubes. They can also include nanotubes which are functionalized by adding chemical groups to their outside surfaces, or inside, or can have chemical groups intercalated (i.e. located between carbon layers).

Preferably the structures or asperities are aligned and perpendicular to the substrate as shown in FIG. 6. The nano-structures are robustly affixed to the electrode substrate 620 to remain attached during ablation, coagulation and other operating procedures. The nanotubes remain sufficiently intact despite being rubbed on tissue. Additionally, the nanotubes and structures or asperities are designed to remain effective in the presence of saline, blood, or water vapor. Materials are selected such that the structures are biocompatible, insoluble in a saline environment, and secured to the substrate.

An anchoring film 630 may be utilized to secure the nanostructures to the substrate. The film 630 may be provided to bond, and or align the nano-structures or asperities to the electrode. The film may also preferably alter, namely improve, the work-function of the active electrode.

A number of manufacturing processes may be used to deposit materials and structures on the electrodes including, for example, use of vacuum processors as described in references cited herein. Additionally, U.S. Pat. No. 6,885,022, to Yaniv et al. and U.S. Patent Publication No. 2009/0038820, to Keefer discuss methods for applying carbon nanotubes on an electrode. It is to be understood, however, that although a number of techniques for applying nanotubes are referred to herein, the invention is not so limited. A wide range of manufacturing techniques known to those of ordinary skill in the art may be employed to coat the electrode surface and to anchor and align the nanotubes. Further details and examples of electrosurgical apparatuses and electrodes for discharging electrons are described in U.S. Pat. Nos. 6,254,600; 6,557,559; 7,241,293; 6,592,738; 6,306,277; 4,298,798; 7,633,216; 6,235,615; 6,103,298; 6,885,022; 7,378,074; and WO 99/05692. Additionally, a number of publications describe electric field emissions instruments and processes including, for example, Shi, et al., Organic Electronics, vol. 9, pp. 859-863 (2008); Bhide, et al., J. Phys. D: Appl. Phys. Vol. 3, p. 443 (1970); Anh, et al., IEEE Sensors Journal, vol. 4, pp. 284-287 (2004); Kusunoki, et al., Jpn. J. Appl. Phys., vol. 32, pp. L1695-L1697 (1993); Navarro-Flores, et al., J. Molec. Catalysis A: Chemical, vol. 242, pp. 182-194; Wada, et al., J. Plasma Fusion Res. SERIES, vol. 8, pp. 1366-1369 (2009); Cristea, et al., J. Optoelectronics and Ad. Mat., vol. 5, pp. 511-520 (2003); Li, et al., Appl. Phys. Lett., vol. 88, 253503-1-3 (2006); Wu, et al. Adv. Mat., vol. 16, 1826-1830 (2004); Lu, et al., J. Phys. Chem. C, vol. 113, pp. 9398-9405 (2009); Huang, et al., Adv. Mat., vol. 18, pp. 114-117 (2006); Huang, et al., Adv. Functional Mat., vol. 17, pp. 1966-1973 (2007), Kishi, et al., Chem. Phys., vol. 192, pp. 387-392 (1995); Tung, et al., Nano Lett., vol. 9, pp. 1949-1955 (2009); Jeong, et al., Trans. Nonferrous Metals Soc. China, vol. 19, pp. s280-s283 (2009); Park, et al., Diamond and Related Mat., vol. 14, pp. 2113-2117 (2005); O'Connel, et al., Chem. Phys. Lett., vol. 342, pp. 265-271 (2001); Wang, et al., New J. Phys., vol. 6 p. 15 (2004); Nie, et al. Nano Res., vol. 3, pp. 103-109 (2010); Song, et al., Chem. Vapor Deposition, vol. 12, pp. 375-379 (2006); Park, et al., J. Vac. Sci. Technol. B, vol. 23, pp. 702-706 (2005); Wilkinson, et al., Adv. Mater., vol. 00, pp. 1-5 (2007); Lee, et al., Appl. Surf. Sci., vol. 254, pp. 513-516 (2007); Shi, et al., Rev. Adv. Mater. Sci., vol. 7, 97-107 (2004); Robinson, et al., Appl. Phys. Lett., vol. 87, pp. 061501-1-3 (2005); Wang., et al., J. Phys. Chem. C; vol. 113, pp. 10446-1 0451 (2009); Tung, et al., Nature Nanotechnology, vol. 4, pp. 25-29 (2009); Paulmier, et. al., Thin Solid Films, vol. 515, pp. 2926-2934 (2007); Paulmier, et al., J. Mater. Processing Techn., vol. 208, pp. 117-123 (2008); Lee, et al., Phys. Lett. A, vol. 370, pp. 345-350 (2007); Qi, et al., Adv. Mater., vol. 15, pp. 411-414 (2003).

FIG. 7 illustrates the removal of a target tissue with an electrosurgical probe 50 in accordance with one embodiment of the present invention. As shown, the high frequency voltage is sufficient to convert the electrically conductive fluid (not shown) between the target tissue 502 and active electrode terminal(s) 504 into an ionized vapor layer 512 or plasma. The active electrode 504 may include any combination of the nano structures or asperities described above. Additionally, the active electrode preferably has an enhanced, namely, lowered work function as described herein. As a result of the applied voltage difference between electrode terminal(s) 504 and the target tissue 502 (i.e., the voltage gradient across the plasma layer 512), charged particles 515 in the plasma are accelerated. At sufficiently high voltage differences, these charged particles 515 gain sufficient energy to cause dissociation of the molecular bonds within tissue structures in contact with the plasma field. This molecular dissociation is accompanied by the volumetric removal (i.e., ablative sublimation) of tissue and the production of low molecular weight gases 514, such as oxygen, nitrogen, carbon dioxide, hydrogen and methane. The short range of the accelerated charged particles 515 within the tissue confines the molecular dissociation process to the surface layer to minimize damage and necrosis to the underlying tissue 520.

During the process, the gases 514 will be aspirated through a suction opening and suction lumen to a vacuum source (not shown). In addition, excess electrically conductive fluid, and other fluids (e.g., blood) will be aspirated from the target site 500 to facilitate the surgeon's view. During ablation of the tissue, the residual heat generated by the current flux lines 510 (typically less than 150° C.) between electrode terminals 504 and return electrode 511 will usually be sufficient to coagulate any severed blood vessels at the site. If not, the surgeon may switch the power supply (not shown) into the coagulation mode by lowering the voltage to a level below the threshold for fluid vaporization, as discussed above. This simultaneous hemostasis results in less bleeding and facilitates the surgeon's ability to perform the procedure.

The enhanced electrode functionality of the present invention serves to increase electrical discharge from the active electrode thereby improving ablation performance. Amongst other things, duty cycle may be increased.

Other modifications and variations can be made to the disclosed embodiments without departing from the subject invention. For example, other uses or applications are possible. Similarly, numerous other methods of controlling or characterizing instruments or otherwise treating tissue using electrosurgical probes will be apparent to the skilled artisan. The instruments and methods described herein may be utilized in instruments for various regions of the body (e.g., shoulder, knee, nose, throat, etc.) and for other tissue treatment procedures (e.g., chondroplasty, menectomy, tonsillectomy, etc.). Thus, while the exemplary embodiments have been described in detail, by way of example and for clarity of understanding, a variety of changes, adaptations, and modifications can be made by one skilled in the art without departing from the scope or teaching herein. The embodiments described herein are exemplary only and are not limiting.

What is claimed is:

1. An electrosurgical apparatus for treating tissue at a target site comprising:
a shaft having a distal end and a proximal end,
an active electrode disposed near the distal end; and
a return electrode wherein when a voltage difference is applied between the active electrode and the return electrode in the presence of an electrically conductive fluid a current path is formed therebetween and wherein the active electrode comprises a low-work function surface to enhance electrical discharge activity.

2. The electrosurgical apparatus of claim 1 wherein said low-work function surface further comprises at least one of micro-structures, micro-asperities, nano-asperities, and nano-structures.

3. The electrosurgical apparatus of claim 2 wherein said low-work function surface comprises nano-structures and said nano-structures comprise tubular shapes.

4. The electrosurgical apparatus of claim 3 wherein said nano-structures are disposed in an aligned formation.

5. The electrosurgical apparatus of claim 1 wherein said low-work function surface is biocompatible and insoluble in a saline.

6. The electrosurgical apparatus of claim 1 wherein said low-work function surface has a work function less than or equal to 3 eV.

7. The electrosurgical apparatus of claim 1 wherein said low-work function surface comprises a layer comprising an alkali metal or alkaline earth metal.

8. The electrosurgical apparatus of claim 1 wherein said active electrode is comprised of a metal doped with a rare earth element, thereby forming a low-work function surface.

9. The electrosurgical apparatus of claim 1 wherein said return electrode comprises a high-work function surface for decreasing discharge activity.

10. The electrosurgical apparatus of claim 9 wherein said high-work function material comprises platinum, iridium, osmium, palladium or gold.

11. An electrosurgical apparatus for treating tissue at a target site comprising:
a shaft having a distal end and a proximal end,
an active electrode disposed near the distal end; and
a return electrode wherein when a voltage difference is applied between the active electrode and the return electrode in the presence of an electrically conductive fluid a current path is formed therebetween and wherein the return electrode comprises a high-work function surface to suppress electrical discharge activity.

12. The electrosurgical apparatus of claim 11 wherein said active electrode comprises a surface comprising at least one of micro-structures, micro-asperities, nano-asperities, and nano-structures.

13. The electrosurgical apparatus of claim 12 wherein said nano-structures comprise tubular shapes.

14. The electrosurgical apparatus of claim 12 wherein said active electrode comprises a low-work function layer for increasing discharge activity.

15. The electrosurgical apparatus of claim 14 wherein said low-work function material comprises at least one compound from the group consisting of alkali metals and alkali earth metals.

16. The electrosurgical apparatus of claim 14 wherein said low-work function surface layer is biocompatible and insoluble in a saline.

17. The electrosurgical apparatus of claim 14 wherein said low-work function surface has a work function less than or equal to 3 eV.

18. The electrosurgical apparatus of claim 11 wherein said high-work function material comprises platinum, iridium, osmium, palladium or gold.

19. The electrosurgical apparatus of claim 16 wherein said surface layer comprises barium oxide.

20. The electrosurgical apparatus of claim 11 wherein said return electrode is positioned on the shaft.

21. An electrosurgical apparatus for treating tissue at a target site comprising:
a shaft having a distal end and a proximal end,
an active electrode disposed near the distal end; and
a return electrode wherein when a voltage difference is applied between the active electrode and the return electrode in the presence of an electrically conductive fluid a current path is formed therebetween and wherein said active electrode comprises a surface comprising at least one of micro-structures, micro-asperities, nano-asperities, and nano-structures to enhance electrical discharge activity.

22. The electrosurgical apparatus of claim 21 wherein the active electrode has a low-function surface.

23. The electrosurgical apparatus of claim 22 wherein said low-work function surface comprises nano-structures and said nano-structures comprise tubular shapes.

24. The electrosurgical apparatus of claim 23 wherein said nano-structures are disposed in an aligned formation.

25. The electrosurgical apparatus of claim 21 wherein said low-work function surface is biocompatible and insoluble in a saline.

26. The electrosurgical apparatus of claim 21 wherein said low-work function surface has a work function less than or equal to 3 eV.

27. The electrosurgical apparatus of claim 21 wherein said low-work function surface comprises an element selected from the group consisting of an alkali metal or alkaline earth metals.

28. The electrosurgical apparatus of claim 21 wherein said active electrode is comprised of a metal doped with a rare earth element, thereby forming a low-work function surface.

29. The electrosurgical apparatus of claim 21 wherein said return electrode comprises a high-work function surface for suppressing discharge activity.

30. The electrosurgical apparatus of claim 29 wherein said high-work function material comprises platinum, iridium, osmium, palladium or gold.
